(19) United States
(12) Patent Application Publication
(10) Pub. No.: US 2014/0277392 A1
(43) Pub. Date: Sep. 18, 2014

(54) ELECTROPOLISHING OF ALLOYS CONTAINING PLATINUM AND OTHER PRECIOUS METALS

(71) Applicant: ABBOTT CARDIOVASCULAR SYSTEMS, INC., Santa Clara, CA (US)

(72) Inventor: William E. Webler, JR., San Jose, CA (US)

(73) Assignee: ABBOTT CARDIOVASCULAR SYSTEMS, INC., Santa Clara, CA (US)

(21) Appl. No.: 13/830,096

(22) Filed: Mar. 14, 2013

(51) Int. Cl. A61F 2/82; A61L 31/02

(52) U.S. Cl. CPC A61F 2/82; A61L 31/022
USPC 623/1.34; 205/659

(57) ABSTRACT

Radiopaque cobalt-based alloys having a smooth electropolished surface with rounded edges and methods for electropolishing such alloys. A cobalt-based alloy includes cobalt, chromium, and one or more radiopaque elements. In one embodiment, examples of radiopaque elements include so-called platinum group metals (i.e., platinum, palladium, ruthenium, rhodium, osmium, or iridium). Group 10 elements (i.e., platinum or palladium) are particularly preferred. Because of the presence of the platinum group metal(s), such alloys are generally difficult to electropolish. Electrolyte formulations and methods for electropolishing such alloys are also disclosed.

### BACKGROUND

Intraluminal stents implanted with percutaneous methods have become a standard adjunct to procedures such as balloon angioplasty in the treatment of atherosclerotic disease of the arterial system. Stents, by preventing acute vessel recoil, improve long term patient outcome and have other benefits such as securing vessel dissections.

Intraluminal stents comprise generally tubular-shaped devices which are constructed to hold open a segment of a blood vessel or other anatomical lumen. Intraluminal stents are used in treatment of diseases such as atherosclerotic stenosis as well as diseases of the stomach and esophagus, and for urinary tract applications. Adequate stent function requires a precise placement of the stent over a lesion or site of plaque or other lumen site in need of treatment. Typically, the stent is delivered to a treatment site by a delivery catheter that comprises an expandable portion for expanding the stent within the lumen.

The delivery catheter onto which the stent is mounted may be a balloon delivery catheter similar to those used for balloon angioplasty procedures. In order for the stent to remain in place on the balloon during delivery to the site of damage within a lumen, the stent may be compressed onto the balloon. The catheter and stent assembly is introduced within a patient's vasculature using a guide wire. The guide wire is disposed across the damaged arterial section and then the catheter-stent assembly is advanced over the guide wire within the artery until the stent is directly within the lesion or the damaged section.

The balloon of the catheter is expanded, expanding the stent against the artery wall. The artery is preferably slightly expanded by the expansion of the stent to seat or otherwise fix the stent to prevent movement. In some circumstances during treatment of stenotic portions of the artery, the artery may have to be expanded considerably in order to facilitate passage of blood or other fluid therethrough. In the case of a self-expanding stent, the stent is expanded by retraction of a sheath or actuation of a release mechanism. Self-expanding stents may expand to the vessel wall automatically without the aid of a dilation balloon, although such a dilation balloon may be used for another purpose.

These manipulations are performed within the body of a patient by a practitioner who may rely upon both placement markers on the stent catheter and on the radiopacity of the stent itself. The stent radiopacity arises from a combination of stent material and stent pattern, including stent strut or wall thickness. After deployment within the vessel, the stent radiopacity should allow adequate visibility of both the stent and the underlying vessel and/or lesion morphology under fluoroscopic visualization.

Stents may be precision cut from drawn metallic tubes by a using a laser cutting machine. The drawn ID/OD surfaces of the metallic tubes are often rough and laser cutting a stent from them produces sharp edges on the stent struts. Typically, the tube wall thickness is greater than desired for the finished stent and the stent struts are cut wider than desired for the finished stent. Typically, the cut stent is electropolished to its finished/desired dimensions. Electropolishing smoothes the ID/OD surfaces of the stent and rounds the edges of the stent struts. Smooth ID/OD surfaces and rounded strut edges make the stent less traumatic to the vessel during stent positioning and deployment and also, minimizes possible damage to the catheter, balloon and/or sheath during their construction and use.

### SUMMARY

Embodiments described herein are directed to articles fabricated from radiopaque cobalt-based alloys having a smooth electropolished surface with rounded edges and methods for electropolishing such alloys. In an embodiment, a cobalt-based alloy includes cobalt, chromium, and one or more radiopaque elements. Examples of radiopaque elements include so-called platinum group metals (i.e., platinum, palladium, ruthenium, rhodium, osmium, or iridium). Group 10 elements (i.e., platinum or palladium) are particularly preferred. Because of the presence of the platinum group metal(s), such alloys are generally difficult to electropolish to attain a smoothed surface with rounded edges.

In an embodiment, a radiopaque body having an electropolished surface and rounded edges is disclosed. The radiopaque body may be formed from a cobalt-based alloy comprising from about 18 weight percent (wt %) to about 39 wt % cobalt, from about 10 wt % to about 25 wt % chromium, from about 20 wt % to about 65 wt % platinum, and wherein the cobalt-based alloy is substantially free of molybdenum. In one embodiment, the electropolished surface is defined as being a smoother electropolished surface. In one embodiment, the edges are defined as being rounded.

In another embodiment, a method for electropolishing a metallic body is disclosed. The method includes (1) positioning the metallic body in an electropolishing electrolyte solution in an electropolishing cell, wherein the electropolishing electrolyte includes one or more of HCl, H₂SO₄, or H₃PO₄, and one or more thickening agents, and (2) electropolishing the metallic body in the electropolishing electrolyte solution in the electropolishing cell, wherein the electropolishing includes an alternating current with a forward:reverse voltage ratio of about 2 and the forward and reverse pulses each having a duration in a range of about 0.003 to about 0.010 seconds.

In yet another embodiment, another method for electropolishing a radiopaque alloy that contains cobalt, chromium, and platinum is disclosed. The method includes (1) positioning the radiopaque body in an electropolishing electrolyte solution in an electropolishing cell, wherein the electropolishing electrolyte includes sulfuric acid (H₂SO₄), hydrochloric acid (HCl), phosphoric acid (H₃PO₄), a thickening agent selected from the group consisting of ethylene glycol, 2-butoxyethanol, glycerol, polyethylene glycol, and one of water or a C₁-C₄ alcohol, and (2) electropolishing the radiopaque body in the electropolishing electrolyte solution in the electropolishing cell, wherein the electropolishing includes an alternating current with a forward:reverse voltage ratio of about 2 and the forward and reverse pulses each having a duration in a range of about 0.003 to about 0.010 seconds.

In the methods described above, the forward voltage may be in a range of about 10 volts to about 25 volts and the reverse voltage may be in a range of about 5 volts to about 12.5 volts. Likewise, an electropolishing run may include about 6,000 to 15,000 forward:reverse voltage cycles or more.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present disclosure, a more particular description of the embodiments of the invention will be rendered by reference to specific embodiments thereof which are illustrated in the appended drawings. It is appreciated that these drawings depict only illustrated embodiments of the disclosure and are therefore not to be considered limiting of its scope. The embodiments of the invention will be described and explained with additional specificity and detail through the use of the accompanying drawings in which:

FIG. 1 is a schematic illustrating an electropolishing apparatus suitable for practicing the electropolishing embodiments described herein;

FIGS. 2A and 2B are schematic cross-sectional views illustrating the effect of electropolishing on surface finish;

FIG. 3A is an isometric view of a stent according to an embodiment of the present disclosure;

FIG. 3B is a plan view of a closure element according to an embodiment of the present disclosure;

FIG. 3C is a side elevation view, in partial cross-section, of a delivery catheter within a body lumen having a stent disposed about the delivery catheter according to an embodiment of the present disclosure; and

FIG. 3D depicts a longitudinal plan view of an embodiment of an expanded embolic protection device, including expandable struts.

### DETAILED DESCRIPTION

### I. Introduction

Embodiments described herein are directed to articles fabricated from radiopaque cobalt-based alloys having a smoother electropolished surface with rounded edges and methods for electropolishing such alloys. In an embodiment, a cobalt-based alloy includes cobalt, chromium, and one or more radiopaque elements. Examples of radiopaque elements include so-called platinum group metals (i.e., platinum, palladium, ruthenium, rhodium, osmium, or iridium). Group 10 elements (i.e., platinum or palladium) are particularly preferred. Because of the presence of the platinum group metal(s), such alloys are generally difficult to electropolish. For example, in the alloys described herein, the application of voltage in an electropolishing electrolyte will often tend to dissolve (ionize) the other metal atoms on the surface quickly, leaving the platinum group metal(s) atoms behind to form an unreactive surface without enough material removal to effectively smooth the surface or round the edges and/or produce a rough, etched surface and not a smoother electropolished surface. In contrast, the electropolishing methods described herein provide electropolished metal articles having substantially improved surface quality, uniformity, rounded edges and a smoother surface finish.

A schematic of a typical electropolishing apparatus 10 suitable for practicing the electropolishing embodiments described herein is illustrated in FIG. 1. The typical electropolishing apparatus 10 includes an electrolyte reservoir 20 that is configured to hold an electropolishing electrolyte solution 40. The typical electropolishing apparatus 10 further includes one or more conductors 60a, 60b, and 70, and a pulse power supply 30 capable of delivering an alternating voltage in short pulses.

In the typical electropolishing apparatus 10, a number of metal work pieces 80 (e.g., stents) are electrically connected to a first terminal 50a of the power supply 30 via conductor 70, while the second terminal 50b of the pulse reverse power supply 30 is connected to conductors 60a and 60b. For convenience sake, the terminal 50a may be referred to as the anode and terminal 50b may be referred to as the cathode, although under an alternating current the polarity of terminals 50a and 50b may change.

The conductors 60a, 60b, and 70 are connected to the pulse reverse power supply 30 and suspended in the reservoir 20 in the electrolyte solution 40. The conductors 60a, 60b, and 70 are submerged in the solution, forming a complete electrical circuit with the electropolishing electrolyte solution 40. An alternating current is applied to the conductors 60a, 60b, and 70 to initiate the electropolishing process.

In the electropolishing methods described herein, for example, electropolishing is carried out with the electropolishing electrolyte solution 40 at a temperature in a range of about −10° C. to about room temperature (e.g., about 22° C.). As such, as further illustrated in FIG. 1, the electropolishing apparatus 10 may also include a combined temperature probe/heating and cooling unit 90, which is attached to a control unit 100. In the illustrated embodiment, the combined temperature probe/heating and cooling unit 90 is submerged in the electropolishing electrolyte solution 40. The control unit 100 may be programmed to monitor and control the temperature of the electropolishing electrolyte solution 40. Other configurations for monitoring/controlling the temperature of the electropolishing electrolyte solution 40 may be used in other embodiments.

The electropolishing apparatus 10 may also include a magnetic stir plate 120 and a magnetic stir bar 110 for mixing the electropolishing electrolyte solution 40 and ensuring even distribution of the electrolyte 40 around the workpieces 80 and the electrodes 60a, 60b, and 70. Other configurations for mixing the electropolishing electrolyte 40 may be used in other embodiments.

For a given electropolishing electrolyte solution, the quantity of metal removed from the work piece is proportional to the amount of current applied and the time. Other factors, such as the geometry of the work piece, affect the distribution of the current and, consequently, have an important bearing upon the amount of metal removed in local areas. For example, FIGS. 2A and 2B illustrate a surface 200 and 230 before and after electropolishing. Sharp regions, such as burrs and sharp edges, illustrated at 210 in FIG. 2A have higher current density than smoother areas illustrated at 220, which leads to the preferential removal of material from the sharp regions 210 and relatively little material removal from the smoother regions. The principle of differential rates of metal removal is important to the concept of deburring accomplished by electropolishing. Fine burrs have very high current density and are, as a result, rapidly dissolved. Smoother areas have lower current density and, as a result, less material is removed from these areas. The result of electropolishing is illustrated in FIG. 2B. As can be seen, the sharp regions illustrated at 210 in FIG. 2A are eroded away leaving a substantially flat, defect free surface 230.

In the course of electropolishing, the work piece is manipulated to control the amount of metal removal so that polishing is accomplished and, at the same time, dimensional tolerances are maintained. Electropolishing literally dissects the metal crystal atom by atom, with rapid attack on the high current density areas and lesser attack on the low current density areas. The result is an overall reduction of the surface profile with a simultaneous smoothing and brightening of the metal surface.

Electropolishing produces a number of favorable changes in a metal work piece (e.g., a stent). These favorable changes include, but are not limited to, one or more of:
Brightening
Burr removal
Oxide and tarnish removal
Reduction in surface profile
Removal of surface occlusions
Increased corrosion resistance
Improved adhesion in subsequent plating
Removal of directional (draw) lines
Radiusing of sharp edges, sharp bends, and corners
Reduced surface friction
Stress relieved surface

### II. An Article Having an Electropolished Surface

Electropolishing is a very effective technique for producing bright, smooth, often approaching mirror-like surfaces on many metals. The technique uses anodic dissolution of prominences on the part being treated, using a suitable electrolyte. Surface finishes with roughness values of 0.1 to 0.01 μm Ra (Ra is the arithmetical average of roughness) may be obtainable on stainless steel, for example.

The electropolishing of stainless steels is not related to precious metals, admittedly; but it is worthwhile to note the reasons as to why the process is undertaken. Improved aesthetics are a major attraction, plus lower labor costs and the ability to polish many areas difficult or impossible to reach when mechanical polishing is used, such as inside complex tubular components. Additionally, it is recognized that these highly-polished surfaces are easier to maintain in a high state of cleanliness. For instance, numerous studies have shown, for example, that bacteriological contamination levels are invariably lower on electropolished surfaces. In the medical device field, devices having a smooth electropolished surface are generally more biocompatible, easier and less traumatic to navigate through interior anatomy, and more corrosion and crack resistant. Electropolishing is also used to remove excess material and to achieve the final dimensions of devices such as stents.

In many applications where precious metals and/or platinum group metals are used, mechanical polishing is often used, but carries a risk of embedded surface contamination. As precious metal-surfaced materials are used for various purposes within the human body, creation of a smoother and purer surface might be particularly desirable. However, the extreme corrosion resistance/unreactivity of platinum group metals makes it difficult to effectively electropolish such metals and metal alloys.

It is, however, relatively easy to dissolve such metals in some corrosive solutions. For example, aqua regia (a mix of hydrochloric and nitric acids) is famous for its ability of dissolve gold, platinum and other precious metals. However, a distinction must be made between etching or dissolving a metal and electropolishing. Etching or dissolving produces a rough surface and is generally incapable of producing a fine, smooth finish. In contrast, electropolishing is, in many cases, capable of producing a reflective, mirror-like finish (e.g., having a sub-micron average roughness of 0.9 to 0.01 μm Ra).

Therefore, in an embodiment, a radiopaque body having an electropolished surface is disclosed. The radiopaque body may be formed from a cobalt-based alloy comprising from about 18 weight percent (wt %) to about 39 wt % cobalt, from about 10 wt % to about 25 wt % chromium, from about 20 wt % to about 65 wt % platinum, and wherein the cobalt-based alloy is substantially free of molybdenum. In one embodiment, the electropolished surface is defined as being a smoother electropolished surface, as opposed to a rough, etched surface or drawn tube surface. In one embodiment, a smooth surface may be defined as a surface having a sub-micron average roughness (e.g., an average roughness of 0.9 to 0.01 μm Ra).

In one embodiment, the wherein the radiopaque body comprises at least a portion of a medical device. Suitable examples of medical devices include, but are not limited to, stents, guidewires, embolic protection filters, and closure elements.

In one embodiment, the radiopaque body having the electropolished surface is electropolished by a method that includes (1) positioning the radiopaque body in an electropolishing electrolyte solution in an electropolishing cell, wherein the electropolishing electrolyte includes one or more of HCl, H₂SO₄, or H₃PO₄, and one or more thickening agents, and (2) electropolishing the radiopaque body in the electropolishing electrolyte solution in the electropolishing cell, wherein the electropolishing includes an alternating current with a forward:reverse voltage ratio of about 2 and the forward and reverse pulses each having a duration in a range of about 0.003 to about 0.010 seconds. A forward voltage is defined as a state where the radiopaque body is positive relative to the cathode, and the reverse voltage is defined as a state where the radiopaque body is negative relative to the cathode.

In one embodiment, the cobalt-based alloy may further include from about 5 wt % to about 12 wt % iron. In one embodiment, the cobalt-based alloy is a quaternary alloy consisting essentially of cobalt, chromium, platinum, and iron. In one embodiment, the cobalt-based alloy is a ternary alloy consisting essentially of cobalt, chromium, and platinum.

In one embodiment, the cobalt-based alloy may further include one or more platinum group metals selected from the group consisting of palladium, rhodium, iridium, osmium, ruthenium, silver and gold. In one aspect, the one or more platinum group metals selected from the group consisting of palladium, rhodium, iridium, osmium, ruthenium, silver and gold may substitute for platinum in the alloy or they may be added in addition to the platinum.

Cobalt is an allotropic elemental material and at temperatures up to 422° C. has a hexagonal close-packed (ε-Co, HCP) crystalline structure, while above 422° C. it has a face center cubic (α-Co, FCC) crystalline structure. The ε-HCP microstructure is relatively brittle and prevents appreciable cold working in the material, while the α-FCC, or austenitic cobalt, is more ductile and allows for cold and hot working for processing purposes. As cobalt is alloyed with other elements, the temperature of the ε-Co to α-Co transformation will increase or decrease based on the microstructure, bonding valence, electronegativity, and atom size of the alloying element. The response of each element with cobalt is shown in their associated binary phase diagrams. If the transformation temperature decreases with the addition of the alloying element there is an enlarged a field and these elements are considered FCC stabilizers. FCC stabilizers include Al, B, Cu, Ti, Zr, C, Sn, Nb, Mn, Fe, and Ni. α-FCC/austenitic stabilization allows for the material to be hot and cold worked during processing from ingot to medical device (e.g., stent).

If the transformation temperature increases with the addition of the alloying element, then there is a restricted α field and these elements are considered HCP stabilizers. HCP stabilizers include Si, Ge, Ar, Sb, Cr, Mo, W, Ta, Re, Ru, Os, Rh, Ir, and Pt. In some Co binary alloys, the transition temperature increases for ε-Co→α-Co, while the transformation temperature for α-Co→ε-Co decreases, which makes the transformation more sluggish, and these elements are considered to have a combined stabilization effect (i.e., they may stabilize both FCC and HCP phases, with the phase present at any given condition being stabilized to retard change to the other phase). Elements with a combined effect on the transformation temperature in cobalt include: Be, Pb, V, Pd, Ga, Au.

Furthermore, as described above, there is a desire to increase the radiopacity of the stent material with addition of platinum group metals, refractory metals, and/or precious metals, such as silver (Ag), gold (Au), hafnium (Hf), iridium (Ir), molybdenum (Mo), palladium (Pd), platinum (Pt), rhenium (Re), rhodium (Rh), tantalum (Ta), tungsten (W), and/or zirconium (Zr), which are more radiopaque than nickel. Radiopaque elements such as Au, Ir, Pd, Pt, Re, Ta, and W all significantly improve the material as their relative radiopacity is at least four times higher than nickel; however, none of these elements are, strictly speaking, FCC stabilizers (at least to the degree of stabilization provided by Ni). Therefore, an increase in concentration of one or more FCC austenitic stabilizers may be important for the workability of an alloy material aimed at increasing the radiopacity and limiting nickel content.

Manganese (Mn) and iron (Fe) are both FCC stabilizers of cobalt based alloys. Increased amounts of Fe have the potential to increase the magnetic response of the material, which may not be desirable in an implantable medical device, although relatively low levels of iron as described herein may be suitable for use. Any of the above described platinum group elements, refractory metal elements, and/or precious metals (e.g., particularly Au, Pd, Pt, Ta, and/or W) could partially or completely replace the Ni content to provide increased radiopacity. As platinum and palladium are in the same periodic table group as nickel they may be particularly good choices to replace nickel to increase radiopacity. At the same time, the concentration of Mn and/or Fe may be increased to provide sufficient FCC stabilization. Target ranges of Mn and/or Fe are dependent upon the desired level of relative radiopacity and governed by solidification dynamics between the elements.

In addition to Mn and Fe as austenitic stabilizers, maintaining some Ni as a stabilizer is also an option explored in some of the examples below. Also as described above, it is desirable that relatively brittle intermetallics that may form between two or more of the components be avoided.

In an embodiment, the manganese may be present from 1 percent to about 25 percent by weight, from about 1 percent to about 17 percent by weight, or from about 1 percent to about 10 percent by weight.

In another embodiment the manganese and any nickel (i.e., Mn+Ni) may be present from 1 percent to about 25 percent by weight, from about 1 percent to about 17 percent by weight, or from about 1 percent to about 10 percent by weight.

In another embodiment the manganese, iron, and any nickel (i.e., Mn+Fe+Ni) may be present from 1 percent to about 25 percent by weight, from about 1 percent to about 17 percent by weight, or from about 1 percent to about 10 percent by weight.

In one embodiment, the cobalt-based alloy is substantially free of tungsten. In one embodiment, the cobalt-based alloy is entirely free of nickel.

Further discussion of and variations on the cobalt-based alloys disclosed herein can be found in U.S. Patent Publication No. 2012/0123525, the entirety of which is incorporated herein by reference.

### III. Methods for Electropolishing a Metallic Body

In an embodiment, a method for electropolishing a metallic body is disclosed. The method includes (1) positioning the metallic body in an electropolishing electrolyte solution in an electropolishing cell, wherein the electropolishing electrolyte solution comprises hydrochloric acid (HCl) and a suitable thickening agent, and (2) electropolishing the metallic body in the electropolishing electrolyte solution in the electropolishing cell, wherein the electropolishing includes an alternating current with a forward:reverse voltage ratio of about 2 and the forward and reverse pulses each having a duration in a range of about 0.003 to about 0.010 seconds. Suitable examples of thickening agents may include, but are not limited to, ethylene glycol, 2-butoxyethanol, glycerol, polyethylene glycol (e.g., PEG 3K), and combinations thereof.

In the methods described above, a forward voltage is defined as a state where the radiopaque body is positive relative to the cathode, and the reverse voltage is defined as a state where the radiopaque body is negative relative to the cathode. In the methods described above, the forward voltage may be in a range of about 10 volts to about 25 volts and the reverse voltage may be in a range of about 5 volts to about 12.5 volts. Likewise, an electropolishing run may include about 6,000 to 15,000 forward:reverse voltage cycles or more. In one embodiment, electropolishing includes 1 to 5 electropolishing runs. The electropolishing in the methods described above may be conducted at a temperature of about −10° C. to about 22° C.

In an embodiment, the electropolishing electrolyte solution comprises hydrochloric acid (HCl) and at least one additional mineral acid, with the proviso that the at least one additional mineral acid does not include nitric acid, and a suitable thickening agent. In another embodiment, the electropolishing electrolyte solution includes sulfuric acid (H₂SO₄), hydrochloric acid (HCl), phosphoric acid (H₃PO₄), a thickening agent, and one of water or a C₁-C₄ alcohol. In one embodiment, the ratio of H₂SO₄ to HCl to H₃PO₄ is in a range of about 3:3:5 to about 2:2:3. In another embodiment, the electropolishing electrolyte solution may include about 3 to 10 parts an H₂SO₄ reagent, about 3 to 10 parts of an HCl reagent, about 5 to 15 parts of an H₃PO₄ reagent, about 5 to 20 parts of the thickening agent.

It should be noted that H₂SO₄, HCl, and H₃PO₄ reagents are typically not pure. I.e., they contain some solvent typically water or another solvent such as methanol. Likewise, the thickening agent may include some solvent (e.g., water or methanol). For example, H₂SO₄ reagent is typically about 95-98% pure, HCl, which is a gas in its pure state, is about 36.5-38% pure as a reagent with the balance being solvent (typically water or methanol), and H₃PO₄ is about 85% pure. A thickening agent like ethylene glycol is usually about 99% pure.

In another embodiment, a method for electropolishing a radiopaque alloy that contains cobalt, chromium, and platinum is disclosed. The method includes (1) positioning the radiopaque body in an electropolishing electrolyte solution in an electropolishing cell, wherein the electropolishing electrolyte includes sulfuric acid (H₂SO₄), hydrochloric acid (HCl), phosphoric acid (H₃PO₄), a thickening agent selected from the group consisting of ethylene glycol, 2-butoxyethanol, glycerol, polyethylene glycol, and one of water or a C₁-C₄ alcohol, and (2) electropolishing the radiopaque body in the electropolishing electrolyte solution in the electropolishing cell, wherein the electropolishing includes an alternating current with a forward:reverse voltage ratio of about 2 and the forward and reverse pulses each having a duration in a range of about 0.003 to about 0.010 seconds.

In one embodiment, the radiopaque alloy may include from about 18 wt % to about 39 wt % cobalt, from about 10 wt % to about 25 wt % chromium, from about 20 wt % to about 65 wt % platinum, and wherein the cobalt-based alloy is substantially free of molybdenum.

In one embodiment, the cobalt-based alloy may further include from about 5 wt % to about 12 wt % iron. In one embodiment, the cobalt-based alloy is a quaternary alloy consisting essentially of cobalt, chromium, platinum, and iron. In one embodiment, the cobalt-based alloy is a ternary alloy consisting essentially of cobalt, chromium, and platinum. For example, the radiopaque alloy may include from about 36 wt % to about 38 wt % cobalt, from about 23 wt % to about 25 wt % chromium, from about 26 wt % to about 28 wt % platinum, and about 10 wt % to about 12 wt % iron.

In one embodiment, the cobalt-based alloy may further include one or more platinum group metals selected from the group consisting of palladium, rhodium, iridium, osmium, ruthenium, silver and gold. In one aspect, the one or more platinum group metals selected from the group consisting of palladium, rhodium, iridium, osmium, ruthenium, silver and gold may substitute for platinum in the alloy or they may be added in addition to the platinum.

In one embodiment, the cobalt-based alloy is substantially free of tungsten. In one embodiment, the cobalt-based alloy is entirely free of nickel.

In one embodiment, the ratio of H₂SO₄ to HCl to H₃PO₄ is in a range of about 3:3:5 to about 2:2:3. In another embodiment, the electropolishing electrolyte solution may include about 3 to 10 parts an H₂SO₄ reagent, about 3 to 10 parts of an HCl reagent, about 5 to 15 parts of an H₃PO₄ reagent, about 5 to 20 parts of the thickening agent.

### IV. Cobalt Alloy Products

As discussed above, the disclosed electropolishing solutions and methods are particularly suitable for electropolishing articles fabricated from the cobalt-based alloys discussed herein. Suitable examples of medical devices that may be fabricated from the cobalt-based alloys disclosed herein and which may be electropolished by the methods disclosed herein include, but are not limited to, stents, guidewires, embolic protection filters, and closure elements.

FIG. 3A is an isometric view of a stent 300 made from a radiopaque cobalt-based alloy according to an embodiment of the present disclosure. The stent 300 includes a stent body 310 sized and configured to be implanted and deployed into a lumen of a living subject. The stent body 310 may be defined by a plurality of interconnected struts 320 configured to allow the stent body 310 to radially expand and contract. However, it is noted that the illustrated configuration for the stent body 310 is merely one of many possible configurations, and other stent-body configurations made from the inventive radiopaque cobalt-based alloy products disclosed herein are encompassed by the present disclosure. For example, the struts 320 may be integrally formed with each other as shown in the illustrated embodiment, separate struts may be joined together by, for example, welding or other joining process, or separate stent sections may be joined together.

The stent body 310 may be made in whole or in part from one of the radiopaque cobalt-based alloys discussed herein. For example, the stent body may be fabricated from an alloy that may include from about 18 wt % to about 39 wt % cobalt, from about 10 wt % to about 25 wt % chromium, from about 20 wt % to about 65 wt % platinum, wherein the cobalt-based alloy is substantially free of molybdenum.

Referring still to FIG. 3A, for example, an average thickness “t” of the struts 320 of the stent body 310 in a radial direction may be about 40 μm to about 100 μm, about 60 μm to about 80 μm, about 50 μm to about 90 μm, about 50 μm to about 77 μm, about 53 μm to about 68.5 μm, or about 58 μm to about 63.5 μm, while also exhibiting a desirable combination of strength, ductility, and radiopacity. Because the disclosed radiopaque cobalt-based alloys are sufficiently strong, the average thickness “t” of the struts 320 of the stent body 310 may be made sufficiently thin to help reduce vessel injury and enhance deliverability while still having a sufficient radiopacity to be visible in X-ray fluoroscopy and MRI.

In one or more embodiments, the stent body 310 may be etched in an acid (e.g., hydrochloric acid) to remove heat-affected zones associated with forming the struts 320 via laser cutting and then electropolished to improve a surface finish of the stent body 310.

Other medical devices besides stents may employ a radiopaque cobalt-based alloy, such as guide wires, closure elements, pacemaker leads, orthopedic devices, embolic coils, sutures, prosthetic heart valves, mitral valve repair coils, or other medical devices or portions thereof for deploying the foregoing medical devices. For example, FIG. 3B illustrates a closure element 330 (e.g., a staple) made from any of the radiopaque cobalt-based alloys disclosed herein. The closure element 330 includes a body 340 defining an outer perimeter 350, an inner perimeter 360, primary tines 370, and secondary tines 380.

Referring now to FIG. 3C, a guide wire device 500 is shown configured to facilitate deploying a stent (e.g., stent 300). FIG. 3C provides detail about the manner in which the guide wire device 500 may be used to track through a patient's vasculature where it can be used to facilitate deployment of a treatment device such as, but not limited to, stent 300. FIG. 3C illustrates a side elevation view, in partial cross-section, of a delivery 400 having a stent 300 disposed thereabout. The portion of the illustrated guide wire device 500 that can be seen in FIG. 3C includes the distal portion 504, the helical coil section 510, and the atraumatic cap section 520. The delivery catheter 400 may have an expandable member or balloon 402 for expanding the stent 300, on which the stent 300 is mounted, within a body lumen 404 such as an artery.

The delivery catheter 400 may be a conventional balloon dilatation catheter commonly used for angioplasty procedures. The balloon 402 may be formed of, for example, polyethylene, polyethylene terephthalate, polyvinylchloride, nylon, Pebax™ or another suitable polymeric material. To facilitate the stent 410 remaining in place on the balloon 402 during delivery to the site of the damage within the body lumen 404, the stent 300 may be compressed onto the balloon 402. Other techniques for securing the stent 300 onto the balloon 402 may also be used, such as providing collars or ridges on edges of a working portion (i.e., a cylindrical portion) of the balloon 402.

In use, the stent 300 may be mounted onto the inflatable balloon 402 on the distal extremity of the delivery catheter 400. The balloon 402 may be slightly inflated to secure the stent 300 onto an exterior of the balloon 402. The catheter/stent assembly may be introduced within a living subject using a conventional Seldinger technique through a guiding catheter 406. The guide wire 500 may be disposed across the damaged arterial section with the detached or dissected lining 407 and then the catheter/stent assembly may be advanced over the guide wire 500 within the body lumen 404 until the stent 300 is directly under the detached lining 407. The balloon 402 of the catheter 400 may be expanded, expanding the stent 300 against the interior surface defining the body lumen 404 by, for example, permanent plastic deformation of the stent 300.

One or more components of the guide wire device 500 may be fabricated from the radiopaque cobalt-based alloy and electropolished by the methods disclosed herein. For example, one or more of the distal portion 504 of the guide wire 500, the coil section 510, or the atraumatic cap 520 may benefit from the radiopacity of the radiopaque cobalt-based alloy disclosed herein. Likewise, usability (e.g., maneuverability) of the guide wire device 500 may, for example, be enhanced by electropolishing one or more components of the device 500 to a smooth finish according to the methods described herein.

Referring now to FIG. 3D, by way of example, the radiopaque cobalt-based alloys described herein may be employed in fabrication of an embolic protection device 470. Such a device may include a filter assembly 472 and expandable strut assembly 474. The embolic protection device may further include an elongated tubular member 475, within which may be disposed a guide wire 500 for positioning the device within a body lumen. The embolic protection device may include a plurality of longitudinal struts 476 and transverse struts 478 that may be fabricated at least in part from a radiopaque cobalt-based alloy according to the present disclosure. In addition, other components of the filter assembly may be formed from a radiopaque cobalt-based alloy. As described above, guidewire 500 (including distal end 510 and/or 520) may include or be constructed from a radiopaque cobalt-based alloy.

### V. Example

Stents were laser cut, manually island removed, etched in an Aqua Regia solution to about 90% of their original (dirty) weight, sonicated in 40° C. deionized water, rinsed in 100% isopropyl alcohol and blown dry prior to electropolishing.

The electrolyte formulations and process conditions identified in this study are as follows: Electrolyte Formulation: the electropolishing electrolyte was formulated as illustrated below in Tables 1 and 2.

TABLE 1

| Component | Parts by Volume |
|---|---|
| 95-98% H₂SO₄ | 3 to 10 |
| 36.5-38% HCI | 3 to 10 |
| 85% H₃PO₄ | 5 to 15 |
| 99% Ethylene Glycol | 2 to 20 |

TABLE 2

| Component | Parts by Volume |
|---|---|
| 95-98% H₂SO₄ | 5 |
| 36.5-38% HCI | 5 |
| 85% H₃PO₄ | 9 |
| 99% Ethylene Glycol | 15 |

Electrolyte Temperature: −10° C. Cathode material: platinum clad Niobium mesh. Anode Clip Material: Nickel. A pulse reverse power supply was used. The forward voltage may be in a range of about 10 volts to about 25 volts and the reverse voltage may be in a range of about 5 volts to about 12.5 volts. An electropolishing run may include about 6,000 to 15,000 forward:reverse voltage cycles or more and a complete electropolishing run for a stent or a similar medical device may include 1 to 5 electropolishing runs.

The electrolyte was in a used condition. That is, the electrolyte contained metal ions and Pt salts from previous electropolishing runs. The electrolyte was a green-blue in color. Test results indicate that this particular formulation may benefit from the presence of these metal ions and Pt salts to produce the desired surface quality. As will be explained in greater detail below, using an electrolyte that is “spiked” with electropolishing product ions had a surprising and unexpected effect on the ability to achieve a smooth surface and rounded edges on articles fabricated from the radiopaque cobalt-based alloys disclosed herein.

The electrolyte and the processing parameters discussed above were identified by a process described below.

It is well-known that a solution containing nitric acid and hydrochloric acid (Aqua Regia) will dissolve platinum without the application of an electropolishing voltage or current (excitation). With the radiopaque cobalt-based alloys discussed herein, testing showed that electropolishing electrolyte solutions containing nitric acid or hydrochloric acid or hydrochloric acid alone will dissolve the radiopaque cobalt-based alloys discussed herein into solution, but only when the applied excitation was AC in nature (consists of alternating positive and negative voltages applied between the stent material and the cathode, both immersed in the electrolyte). DC and DC pulsed applied voltages (currents) were ineffective in causing any significant mass removal. Investigation of nitric acid containing electropolishing electrolyte formulations were unsuccessful, for reasons that will be discussed below, so hydrochloric acid containing solutions were investigated further.

Examination of known processes for electropolishing CoCr and observing development test results indicates that the alloy metals in the radiopaque cobalt-based alloys discussed herein, except for platinum, go into solution (become ionized and dissolve in the electrolyte) during the time that the stent is positively charged relative to the cathode. Also, as a result of the positive charge on the stent and the presence of chlorine ions (from the hydrochloric acid), chlorine gas is formed at the metal surface, some which reacts with the platinum at the metal surface to form an relatively insoluble salt or salt-like platinum compound. This platinum compound deposits onto and covers the metal surface, substantially preventing any further dissolution of or reaction with the metal surface. Thus, the application of a DC electropolishing electrical excitation is ineffective. Simply removing the applied excitation for a period of time did not remove the deposit to a significant or practical extent. Thus, the application of a pulsed DC electropolishing electrical excitation is also ineffective.

With a subsequent reversal of the excitation voltage (reversal of the voltage/electrical current direction), at least a portion of this platinum compound is reacted into a more soluble salt/dissolved and thus, platinum is removed from the metal surface and the metal surface is uncovered, such that another subsequent excitation reversal may start the dissolution cycle again. Thus, an AC excitation voltage is effective for dissolving the radiopaque cobalt-based alloys discussed herein in a hydrochloric acid containing electrolyte. It is also likely that some of the radiopaque cobalt-based alloy metals, including platinum, are also deposited on the metal surface during this time that the stent/metal surface is negatively charged relative to the cathode.

Testing of the most cost effective electropolishing formulations for platinum, platinum alloys and CoPt alloys in the discovered literature showed that they provided an etched-like surface finish and not the desired smooth electropolished finish. One of the conclusions based on review of these data was that it is relatively easy to find a solution that will dissolve the alloys discussed herein, but that the know formulations and their associated processing parameters provide an etched-like surface finish and not the desired smooth electropolished finish.

These and other data indicated the possibility that the electropolishing boundary layer of these formulations was too thin to produce the desired electropolished surface finish. The electropolishing boundary layer is a resistive region (resistive to the introduction of metal ions and other electropolishing process produced species) at the metal surface and in which the electrolyte is saturated with metal ions and other electropolishing process produced species (ions, elements or compounds).

In accordance with the present disclosure, the following parameters were varied in order to thicken the electropolishing boundary layer. It was found that thickening the boundary layer could allow the formation of a smooth electropolished surface by altering the electropolishing conditions in the following ways:

1. Vary Forward:Reverse AC Pulse Duration and Vary Amplitude.

As discussed above, application of alternating current was necessary in order erode the alloys discussed herein in the disclosed electrolytes. However, pulse duration, ratio of the forward and reverse pulses, and their amplitude have a large impact on the thickness of the boundary layer. It was found, for example, that during the reverse pulse phase the boundary layer tends to dissipate and that the forward pulse tends to reestablish the boundary layer. Thus, it was believed that shorter pulses would help maintain the boundary layer. Testing was begun at 0.2 second pulses. It was found that such pulses did not yield a smooth electropolished surface and, as a result, pulse duration was systematically adjusted downward from there.

For reasons that will be discussed below, it is desirable to maximize the mass removal rate. Test results indicate that the rate of dissolution (sample mass removal rate) is greatly affected by the amplitudes, amplitude ratio and period/duration of the AC (pulse reverse) excitation. For the electropolishing solutions discussed herein, variations of the disclosed electrolyte formulation using rods or stents and a pulse reverse power supply (Dynatronix DPR 40-15-30), the results were very consistent. A forward (stent positive relative to the cathode) to reverse (stent negative relative to the cathode) voltage amplitude ratio of about 1:1 to about 3:1 (e.g., 2:1) provided the maximum mass removal rate. Pulse durations in the range of 0.003 to 0.010 seconds provided the maximum mass removal rates. In theory, mass removal rate may be increased by using shorter pulses (e.g., about 0.001 seconds or less) while simultaneously increasing the reverse voltage amplitude (going to a lower forward to reverse voltage amplitude ratio), but equipment capable producing such short pulses is not readily available and there may be safety issues associated with using higher voltages.

2. Increase the Viscosity of the Electrolyte.

A higher viscosity electrolyte lowers the diffusion rate of metal ions and other electropolishing produced species out of the boundary layer and the diffusion rate of reactive species into the boundary layer. Thus, for any given rate of metal ion and other species production at the surface of the metal, at a higher viscosity, a greater boundary layer surface area is required to support that rate of diffusion out of and into the boundary layer, which requires a greater boundary layer thickness.

During the development process for the electrolyte and process parameters discussed herein, the ethylene glycol content of the formulation was increased to increase the viscosity of the electrolyte. The feasibility of the current electrolyte and the associated process parameters were first demonstrated by systematically increasing the viscosity of the electrolyte. There are many compounds besides ethylene glycol that can be used to increase electrolyte viscosity (for example, 2-n-butoxyethanol, glycerol, polyethylene glycol (PEG), etc.). Formulations that contain nitric acid generally can't have their viscosities adjusted because nitric acid reacts with organic compounds to form dangerous and/or explosive compounds, which is why nitric acid was excluded from the formulations discussed herein.

3. Lower the Temperature of the Electrolyte.

The lower the temperature of the electrolyte, the slower diffusion occurs and thus, the same description of boundary layer thickening as in 1 and 2 above is applicable. Additionally, at lower temperatures, fluids become more viscous. It is preferred to have electrolyte effective electropolishing temperatures at or just above room temperature (for temperature control/mass loss rate control purposes). Low temperatures require bulky equipment for cooling and can cause water to condense out of the air into the electrolyte, which can interfere with the electropolishing process in several ways. However, high temperatures can cause the loss (evaporational fuming) of electrolyte components, which also can interfere with the electropolishing process. Despite this, many commercial electropolishing electrolytes are operated at elevated or very low temperatures.

4. Increase the Rate of Metal Dissolution/Mass Loss Rate (Create Metal Ions and Other Electropolishing Produced Species at a Greater Rate).

There are several ways to cause this rate to increase:

a. Increase the Electrolyte Temperature.

This fights against 3 above and facilitates b below and thus, requires testing to determine which effects will be dominant/most beneficial. Analysis of development test results indicates that temperature increases result in significant increases in mass loss rates, on the order of a 150% increase in mass loss rate per 10° C. increase in the −10° to 22° C. range, all other conditions being equal.

b. Increase the Acid (H⁺) Availability of the Electrolyte Formulation.

This can fight against 2 and 3 above. Formulations often contain several acids and thus, increased (Fr) availability can be obtained by increasing the content of the strongest acid(s). Additionally, one may choose to increase the content of the more viscous strong acid.

c. Increase the Electropolishing Voltage/Current Amplitudes.

Naturally, there are practical limits to how high an electropolishing voltage/current can be increased before you run into problems. Most commonly, formulations containing water are limited by the production of oxygen bubbles on the metal surface, which interferes with electropolishing under the bubble. Additionally, formulations containing HCl can be limited by the production of chlorine bubbles on the metal surface for the same reason. However, in this invention, the production of some chlorine gas, which may form bubbles, is likely necessary to react with the platinum and aid in its dissolution process. Surfactants can be added to the formulation to lower the surface tension and cause bubbles to be less likely to adhere to the metal surface. Additionally, high currents can lead to heating of the stent and/or the electrolyte that can cause unwanted bubbles, melts/discoloration of the stent and a lack of control of the electropolishing process due to a lack of electrolyte temperature control.

5. Increase the Amount of Metal Ions and Other Electropolishing Produced Species in the Bulk of the Electrolyte.

This decreases the concentration gradient at the electrolyte surface of the boundary layer and thus decreases the rate of diffusion of metal ions and other electropolishing produced species out of the boundary layer and the same description of boundary layer thickening as in 2 above is applicable. Additionally, fewer metal ions and other electropolishing produced species are required to saturate the solution (form a boundary layer) at the metal surface. This method is not very desirable for several reasons, but it is a feature common to many electropolishing solutions/processes and is often called “spiking.” Spiking can involve adding used electrolyte to new electrolyte or electropolishing “sacrificial” material into the new electrolyte prior to electropolishing your parts or devices.

6. Decrease the Solubility of the Electrolyte to the Metal Ions of Interest.

Thus, the boundary layer becomes more easily saturated (requires fewer metal ions per unit volume to reach saturation) and the boundary layer must thicken. In this invention, Co, Cr, and Fe ions are soluble in water. Ions tend to be less soluble in less polar fluids, fluid/dissolved polar compounds with significant organic portions. Thus, incorporating or increasing the amount of less polar compounds can thicken the boundary layer. This also facilitates 2 above (fluid/dissolved polar compounds with significant organic portions tend to be viscous).

It is important to note that the parameters outlined in points 1-6 above are interconnected and that changes in one may affect another. For instance, by increasing the “spiking” and/or lowering the temperature of the electrolyte, it may be possible to reduce the amount to the thickening agent in the solution while increasing or at least maintaining mass loss rates and while still providing a smooth electropolished surface. It is also important to note that it is possible that too thick a boundary layer will also lead to a poor surface finish, so boundary layer thickness increasing steps should be pursued in stages to avoid missing conditions that produce an acceptable boundary layer thickness (avoid going directly from too thin to too thick a boundary layer). Process development test results indicate that the electropolishing process can be developed further to provide more desirable results by one or more of the following:

I. Test the formulation(s) at temperatures nearer room temperature.

II. Increasing the glycol (and/or use some other more viscous compound) content of the electrolyte formulation.

III. Using methanolic HCl in the formulation instead of “normal” HCl which contains water.

IV. Replacing some or all of the phosphoric acid with glycol or some other more viscous compound.

V. Adjusting the ratio of sulfuric acid to hydrochloric acid.

VI. Start low and increase the applied voltage/current until a good surface is obtained or until bubble or heat generation becomes an issue.

VII. Adjusting the forward to reverse voltage amplitude ratio for maximum mass removal rate.

VIII. Adjusting the forward and/or reverse durations for the maximum mass removal rate.

Another consideration is the time required to electropolish a stent. The greater the required electropolishing time, the lower the throughput capacity for the existing electropolishing equipment/facilities/operators will become. The disclosed formulation requires about 4.25 minutes to electropolish a stent to approximately 70% of its initial/clean weight (a typical mass loss %). This is likely too long. In such a case, it may be desirable to use a high mass loss electrolyte, such as HCl or HCl with ethylene glycol, in order to rapidly remove the bulk of the mass/round sharp edges and then use the electropolishing electrolyte and processing parameters discussed herein to attain the desired final weight and a smooth electropolished surface.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A radiopaque body having an electropolished surface, comprising:
the radiopaque body formed from a cobalt-based alloy comprising from about 18 weight percent (wt %) to about 39 wt % cobalt, from about 10 wt % to about 25 wt % chromium, from about 20 wt % to about 65 wt % platinum, and wherein the cobalt-based alloy is substantially free of molybdenum.

2. The radiopaque body of claim 1, wherein the electropolished surface is a smooth electropolished surface.

3. The radiopaque body of claim 1, wherein the cobalt-based alloy is electropolished by a method that includes:
positioning the radiopaque body in an electropolishing electrolyte solution in an electropolishing cell, wherein the electropolishing electrolyte includes one or more of HCl, H₂SO₄, or H₃PO₄, and one or more thickening agents; and
electropolishing the radiopaque body in the electropolishing electrolyte solution in the electropolishing cell, wherein the electropolishing includes an alternating current with a forward:reverse voltage ratio of about 2 and the forward and reverse pulses each having a duration in a range of about 0.003 to about 0.010 seconds.

4. The radiopaque body of claim 1, wherein the cobalt-based alloy further comprises from about 5 wt % to about 12 wt % iron.

5. The radiopaque body of claim 4, wherein the cobalt-based alloy is a quaternary alloy consisting essentially of cobalt, chromium, platinum, and iron.

6. The radiopaque body of claim 1, wherein the cobalt-based alloy further comprises from about 1 wt % to about 25 wt % manganese.

7. The radiopaque body of claim 1, wherein the cobalt-based alloy further comprises one or more platinum group metals selected from the group consisting of palladium, rhodium, iridium, osmium, ruthenium, silver and gold.

8. The radiopaque body of claim 1, wherein the cobalt-based alloy is substantially free of tungsten.

9. The radiopaque stent of claim 1, wherein the cobalt-based alloy is entirely free of nickel.

10. The radiopaque stent of claim 1, wherein the cobalt-based alloy is a ternary alloy consisting essentially of cobalt, chromium, and platinum.

11. A method for electropolishing a metallic body, comprising:
positioning the metallic body in an electropolishing electrolyte solution in an electropolishing cell, wherein the electropolishing electrolyte includes one or more of HCl, H₂SO₄, or H₃PO₄, and one or more thickening agents; and
electropolishing the metallic body in the electropolishing electrolyte solution in the electropolishing cell, wherein the electropolishing includes an alternating current with a forward:reverse voltage ratio of about 2 and the forward and reverse pulses each having a duration in a range of about 0.003 to about 0.010 seconds.

12. The method of claim 11, wherein the forward voltage is in a range of about 10 volts to about 25 volts and the reverse voltage is in a range of about 5 volts to about 12.5 volts.

13. The method of claim 11, wherein the electropolishing includes an electropolishing run that about 6,000 to 15,000 forward:reverse voltage cycles or more.

14. The method of claim 13, wherein the electropolishing includes 1 to 5 electropolishing runs.

15. The method of claim 11, wherein the electropolishing electrolyte solution in the electropolishing cell is held at a temperature of about −10° C. to about 22° C.

16. The method of claim 11, wherein the electropolishing electrolyte solution includes sulfuric acid (H₂SO₄), hydrochloric acid (HCl), phosphoric acid (H₃PO₄), a thickening agent selected from the group consisting of ethylene glycol, 2-butoxyethanol, glycerol, polyethylene glycol, and combinations thereof, and one of water or a C₁-C₄ alcohol.

17. The method of claim 16, wherein the ratio of H₂SO₄ to HCl to H₃PO₄ is in a range of about 3:3:5 to about 2:2:3.

18. The method of claim 16, wherein the electropolishing electrolyte solution includes about 3 to 10 parts an H₂SO₄ reagent, about 3 to 10 parts of an HCl reagent, about 5 to 15 parts of an H₃PO₄ reagent, about 5 to 20 parts of the thickening agent.

19. The method of claim 18 wherein the HCl is methanolic HCl and the solvent is methanol.

20. The method of claim 11, wherein the metallic body comprises at least a portion of a medical device selected from the group consisting of stents, guidewires, embolic protection filters, and closure elements.

21. A method for electropolishing a radiopaque alloy containing cobalt, chromium, and platinum, the method comprising:
positioning the radiopaque body in an electropolishing electrolyte solution in an electropolishing cell, wherein the electropolishing electrolyte includes sulfuric acid (H₂SO₄), hydrochloric acid (HCl), phosphoric acid (H₃PO₄), a thickening agent selected from the group consisting of ethylene glycol, 2-butoxyethanol, glycerol, polyethylene glycol, and one of water or a C₁-C₄ alcohol; and
electropolishing the radiopaque body in the electropolishing electrolyte solution in the electropolishing cell, wherein the electropolishing includes an alternating current with a forward:reverse voltage ratio of about 2 and the forward and reverse pulses each having a duration in a range of about 0.003 to about 0.010 seconds.

22. The method of claim 21, wherein the radiopaque alloy comprises from about 18 wt % to about 39 wt % cobalt, from about 10 wt % to about 25 wt % chromium, from about 20 wt % to about 65 wt % platinum, and wherein the cobalt-based alloy is substantially free of molybdenum.

23. The method of claim 21, wherein the radiopaque alloy further comprises from about 5 wt % to about 12 wt % iron.

24. The method of claim 23, wherein the radiopaque alloy is a quaternary alloy consisting essentially of cobalt, chromium, platinum, and iron.

25. The method of claim 21, wherein the radiopaque alloy comprises from about 36 wt % to about 38 wt % cobalt, from about 23 wt % to about 25 wt % chromium, from about 26 wt % to about 28 wt % platinum, and about 10 wt % to about 12 wt % iron.

26. The method of claim 21, wherein the cobalt-based alloy further comprises one or more platinum group metals selected from the group consisting of palladium, rhodium, iridium, osmium, ruthenium, silver and gold.

27. The method of claim 21, wherein the ratio of H₂SO₄ to HCl to H₃PO₄ is in a range of about 3:3:5 to about 2:2:3.

28. The method of claim 21, wherein the electropolishing electrolyte solution includes about 3 to 10 parts an H₂SO₄ reagent, about 3 to 10 parts of an HCl reagent, about 5 to 15 parts of an H₃PO₄ reagent, about 5 to 20 parts of the thickening agent.

29. The method of claim 28, wherein the HCl is methanolic HCl.
